(19) United States
(12) Patent Application Publication
(10) Pub. No.: US 2023/0407418 A1
(43) Pub. Date: Dec. 21, 2023

(54) MASSIVELY SCALABLE VIRAL TESTING AND ASYMPTOMATIC SURVEILLANCE

(71) Applicants: CZ Biohub SF, LLC, Stanford, CA (US); The Board of Trustees of the Leland Stanford Junior University, Stanford, CA (US)

(72) Inventors: Sandy L. Klemm, Stanford, CA (US); William J. Greenleaf, Stanford, CA (US)

(21) Appl. No.: 18/248,121

(22) PCT Filed: Oct. 6, 2021
(86) PCT No.: PCT/US2021/053834

§ 371 Date: Apr. 6, 2023

Related U.S. Application Data
(60) Provisional application No. 63/088,855, filed on Oct. 7, 2020.

(51) Int. Cl. C12Q 1/70; C12N 7/00; C12Q 1/6883

(52) U.S. Cl. CPC C12Q 1/701; C12N 7/00; C12Q 1/6883; C12N 2770/20022

(57) ABSTRACT

Described herein is a method of rapidly identifying a patient that is positive for infection with a single-stranded RNA or DNA virus using a massively scalable viral testing method.

### CROSS-REFERENCE TO RELATED APPLICATIONS

This application claims priority to U.S. provisional application No. 63/088,855 filed Oct. 7, 2020, the entire contents of which are incorporated by reference.

### STATEMENT AS TO RIGHTS TO INVENTIONS MADE UNDER FEDERALLY SPONSORED RESEARCH AND DEVELOPMENT

This invention was made with Government support under contract W911NF1920185 awarded by the Defense Advanced Research Projects Agency. The Government has certain rights in the invention.

### SEQUENCE LISTING SUBMITTED IN ASCII FORMAT

This application contains a Sequence Listing that has been submitted electronically in ASCII format and is hereby incorporated by reference in its entirety. Said ASCII copy, created on Oct. 4, 2021, is named 103182-1270065-005910WO_SL.txt and is 11,926 bytes in size.

### BACKGROUND

In order to contain the Covid-19 global pandemic, protect high-risk populations from infection, and sustainably resume economic activity, a significant fraction of the asymptomatic global population must be routinely tested for the causative SARS-CoV-2 virus. Periodic testing of asymptomatic essential workers and students will require a 100-fold expansion of global testing capacity, yet commercial laboratories are currently unable to process even symptomatic patients on clinically relevant time scales, and rapid self-tests are not sufficiently accurate to provide actionable diagnostic information. There is a therefore an urgent need for technological advances that scale clinical-grade viral testing by orders-of-magnitude.

### BRIEF SUMMARY OF THE INVENTION

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the illustrative embodiments of the present invention are discussed below.

The present disclosure provides methods for concurrent sample processing called Identity Preserving Sample Multiplexing (IPSM) that provides the ability to scale SARS-CoV-2 testing by orders of magnitude.

In one aspect, the disclosure provides a method for rapid identification of a SARS-CoV-2 positive subject, the method comprising:
(a) incubating a patient nucleic acid sample comprising RNA obtained from a patient to be evaluated for SARS-CoV-2 infection with an oligonucleotide that comprises a patient-specific identifying sequence that distinguishes the nucleic acid sample from the patient from nucleic acid samples from other patients in a pool of patient nucleic acid samples, wherein incubation comprises annealing SARS-CoV-2 nucleic acid, if present in the patient nucleic acid sample, with at least three collinear oligonucleotides that are reverse complementary to the sense SARS-CoV-2 target sequence under conditions to form a hybridized oligonucleotide-SARS-CoV-2 complex, wherein the at least three collinear oligonucleotides are each hybridized at adjacent positions to the respective target region of the SARS-CoV-2 genome;
(b) pooling the patient nucleic acid sample following incubation in step (a) with a plurality of nucleic acid samples from other patients incubated as in (a), but where the patient-specific identifier sequence is different for each of the other patient samples present in the pool, relative to each of the other patient specific-specific barcodes;
(c) purifying hybridized oligonucleotide-SARS-CoV-2 nucleic acid complexes, when present, from the pool;
(d) ligating the three oligonucleotides hybridized to a SARS-CoV-2 nucleic acid in the oligonucleotide-SARS-CoV-2 complexes, if present, with a DNA ligase that is capable of ligation with an RNA splint to provide ligation products; and
(e) amplifying the ligation products, if present, to produce amplicons and detecting the presence or absence of the amplicon, wherein detection of amplicons in a pooled sample indicate that one or more patient samples comprises SARS-CoV-2 RNA. In some embodiments, the method further comprises (f) performing an asymmetric RNaseH-dependent PCR on a positive pool to provide a library of nucleic acid molecules for sequencing, wherein the asymmetric PCR comprises amplification using patient-specific primers, each of which hybridizes to a patient-specific barcode sequence, and is present in approximately the same limiting concentration; and (g) sequencing the library of nucleic acid molecules to determine the patient-specific identifier sequences, thereby identifying a SARS-CoV-2-positive patient. In some embodiments, the target region of the SARS-CoV-2 viral nucleic acid of each of the collinear oligonucleotides has low secondary structure. In some embodiments, the oligonucleotide has a GC content from about 45% to about 55%. In some embodiments, the amplification reaction of (e) is quantitative PCR. In alternative embodiments, embodiments, the amplification reaction of (e) is rolling circle amplification (RCA) or loop-mediated isothermal amplification (LAMP). In some embodiments, the DNA ligase of (c) is Chlorella virus DNA ligase PBCV-1. In some embodiments, each of the three oligonucleotides has Tm of 55° C. or higher. In some embodiments, the oligonucleotide hybridized in the 5′-most position comprises a patient-specific barcode sequence at the 3′ end; and/or the oligonucleotide hybridized to the 3′-most position comprises a patient-specific barcode. In some embodiments, the oligonucleotide hybridizes to the most 5′ position comprises the patient-specific identifier sequence and further comprises a unique molecular identifier sequence at the 5′ end of the patient-specific identifier sequence. In some embodiments, each of the three oligonucleotides comprise one or more locked nucleic acid monomers. In some embodiments, the 3′-most oligonucleotide is linked at its 5′ end to a purification moiety, such as biotin. In some embodiments, the oligonucleotide that hybridizes to the most 5′ position comprises a region at the 5′ end that is not complementary to the target region of SARS-CoV-2 to which the oligonucleotide binds, but is reverse complementary to the first four nucleotides in the 3′ end that are complementary to the target region of the SARS-CoV-2 target region and form a stem-loop structure in the absence of viral template. In some embodiments, the oligonucleotide that hybridizes in the 5′ position comprises the patient-specific identifier sequence and at least said 5′ most oligonucleotide is present in at least 2-fold molar excess of the SARS-CoV-2 nucleic acid. In some embodiments, the method further comprises a step of incubating the hybridized complex with a 5′ exonuclease after (a) and prior to (b). In some embodiments, the Tm of each of the three collinear oligonucleotides is above 80° C. In some embodiments, the Tm of each of the three collinear oligonucleotides is in the range of 60° C. to 95° C.

In a further aspect, the disclosure provides a method for rapid identification of a SARS-CoV-2 positive subject, the method comprising:
(a) incubating a patient nucleic acid sample comprising RNA obtained from a patient to be evaluated for SARS-CoV-2 infection with an oligonucleotide that comprises a patient-specific identifying sequence at the 5′ end that distinguishes the nucleic acid sample from the patient from nucleic acid samples from other patients in a pool of patient nucleic acid samples, wherein incubation comprises annealing SARS-CoV-2 nucleic acid, if present in the patient nucleic acid sample, with the oligonucleotide, wherein the oligonucleotide comprises a sequence complementary to a target region of the SARS-CoV-2 viral nucleic acid;
(b) pooling the patient nucleic acid sample following incubation in step (a) with a plurality of nucleic acid samples from other patients incubated as in (a), but where the patient-specific identifier sequence is different for each of the other patient samples present in the pool, relative to each of the other patient specific-specific barcodes;
(c) purifying hybridized oligonucleotide-SARS-CoV-2 nucleic acid complexes, when present, from the pool;
(d) performing a reverse transcriptase reaction to extend the oligonucleotide hybridized to the SAR-Co-V-2 nucleic acids; and
(e) performing an amplification reaction on the product obtained in (d), if present, to produce amplicons and detecting the presence or absence of the amplicons to determine whether the pool is positive for the presence of SARS-CoV-2 polynucleotide sequences. In some embodiments, the target region of the SARS-CoV-2 viral nucleic acid has low secondary structure. In some embodiments, the oligonucleotide has a GC content from about 45% to about 55%. In some embodiments, the method further comprises (f) performing an asymmetric RNaseH-dependent PCR on a positive pool to provide a library of nucleic acid molecules for sequencing, wherein the asymmetric PCR comprises amplification using patient-specific primers, each of which hybridizes to a patient-specific barcode sequence, and is present in approximately the same limiting concentration; and (g) sequencing the library of nucleic acid molecules to determine the patient-specific identifier sequences, thereby identifying a SARS-CoV-2-positive patient. In some embodiments, the amplification reaction of (e) is quantitative PCR. In alternative embodiments, embodiments, the amplification reaction of (e) is rolling circle amplification (RCA) or loop-mediated isothermal amplification (LAMP). In some embodiments, the oligonucleotide comprises one or more locked nucleic acid monomers. In some embodiments, the oligonucleotide is linked to a purification moiety, such as biotin. In some embodiments, the oligonucleotide comprises a region at the 5′ end that is not complementary to the target region of SARS-CoV-2 to which the oligonucleotide binds, but is reverse complementary to the first four nucleotides in the 3′ end that are complementary to the target region of the SARS-CoV-2 target region and form a stem-loop structure in the absence of viral template. In some embodiments, the oligonucleotide is present in at least 2-fold molar excess of the SARS-CoV-2 nucleic acid. In some embodiments, the method further comprises a step of incubating the hybridized complex with a 3′ exonuclease prior to (d). In some embodiments, the Tm of the oligonucleotide is above 80° C. In some embodiments, the Tm of the oligonucleotide is in the range of 65° C. to 95° C.

In another aspect, provided herein is a method for rapid identification of a patient that is infected with a single-stranded RNA (ssRNA) virus, the method comprising: (a) incubating a patient nucleic acid sample comprising RNA obtained from a patient to be evaluated for infection with the ssRNA virus with an oligonucleotide that comprises a patient-specific identifying sequence that distinguishes the nucleic acid sample from the patient from nucleic acid samples from other patients in a pool of patient nucleic acid samples, wherein incubation comprises annealing ssRNA nucleic acid, if present in the patient nucleic acid sample, with at least three collinear oligonucleotides that are reverse complementary to the ssRNA target sequence under conditions to form a hybridized oligonucleotide-viral nucleic acid complex, wherein the at least three collinear oligonucleotides are each hybridized at adjacent positions to the respective target region of the ssRNA genome, and wherein each of the three oligonucleotides hybridizes to a target region of the ssRNA viral nucleic acid;
(b) pooling the patient nucleic acid sample following incubation in step (a) with a plurality of nucleic acid samples from other patients incubated as in (a), but where the patient-specific identifier sequence is different for each of the other patient samples present in the pool, relative to each of the other patient specific-specific barcodes;
(c) purifying hybridized oligonucleotide-ssRNA nucleic acid complexes, when present, from the pool;
(d) ligating the three oligonucleotides hybridized to a ssRNA nucleic acid in the oligonucleotide-ssRNA complexes with a DNA ligase that is capable of ligation with an RNA splint to provide a ligation product; and
(e) performing an amplification reaction on a portion of the ligation product obtained in (d) that is capable of detecting ssRNA nucleic acids to determine whether the pool is positive for the presence of ssRNA polynucleotide sequences. In some embodiments, the method further comprises:
(f) performing an asymmetric RNaseH-dependent PCR on a positive pool to provide a library of nucleic acid molecules for sequencing, wherein the asymmetric PCR comprises amplification using patient-specific primers, each of which hybridizes to a patient-specific barcode sequence, and is present in approximately the same limiting concentration; and
(g) sequencing the library of nucleic acid molecules to determine the patient-specific identifier sequences, thereby identifying a patient infected with the ssRNA virus. In some embodiments, the target region of the SARS-CoV-2 viral nucleic acid of each of the collinear oligonucleotides has low secondary structure. In some embodiments, the oligonucleotide has a GC content from about 45% to about 55%. In some embodiments, the amplification reaction of (e) is quantitative PCR. In alternative embodiments, embodiments, the amplification reaction of (e) is rolling circle amplification (RCA) or loop-mediated isothermal amplification (LAMP). In some embodiments, the DNA ligase of (c) is Chlorella virus DNA ligase PBCV-1. In some embodiments, each of the three oligonucleotides has Tm of 55° C. or higher. In some embodiments, the oligonucleotide hybridized in the 5′-most position comprises a patient-specific barcode sequence at the 3′ end; and/or the oligonucleotide hybridized to the 3′-most position comprises a patient-specific barcode. In some embodiments, the oligonucleotide hybridized to the most 5′ position comprises the patient-specific identifier sequence and further comprises a unique molecular identifier sequence at the 5′ end of the patient-specific identifier sequence.

In an additional aspect, the disclosure provides a method of rapid identification of a patient infected with a ssRNA virus, the method comprising: (a) incubating a patient nucleic acid sample comprising RNA obtained from a patient to be evaluated for infection with the ssRNA virus with an oligonucleotide that comprises a patient-specific identifying sequence at the 5′ end that distinguishes the nucleic acid sample from the patient from nucleic acid samples from other patients in a pool of patient nucleic acid samples, wherein incubation comprises annealing ssRNA viral nucleic acid, if present in the patient nucleic acid sample, with the oligonucleotide, wherein the oligonucleotide hybridizes to a target region of the ssRNA viral nucleic acid; (b) pooling the patient nucleic acid sample following incubation in step (a) with a plurality of nucleic acid samples from other patients incubated as in (a), but where the patient-specific identifier sequence is different for each of the other patient samples present in the pool, relative to each of the other patient specific-specific barcodes; (c) purifying hybridized oligonucleotide-ssRNA virus nucleic acid complexes, when present, from the pool; (d) performing a reverse transcriptase reaction to extend the oligonucleotide hybridized to the ssRNA viral nucleic acids; and (e) performing an amplification reaction on a portion of the product obtained in (d) that is capable of detecting ssRNA viral nucleic acids to determine whether the pool is positive for the presence of ssRNA viral polynucleotide sequences. In some embodiments, the method further comprises (f) performing an asymmetric RNaseH-dependent PCR on a positive pool to provide a library of nucleic acid molecules for sequencing, wherein the asymmetric PCR comprises amplification using patient-specific primers, each of which hybridizes to a patient-specific barcode sequence, and is present in approximately the same limiting concentration; and (g) sequencing the library of nucleic acid molecules to determine the patient-specific identifier sequences, thereby identifying a patient that is infected with the ssRNA virus. In some embodiments, the target region of the SARS-CoV-2 viral nucleic acid of each of the collinear oligonucleotides has low secondary structure. In some embodiments, the oligonucleotide has a GC content from about 45% to about 55%. In some embodiments, the amplification reaction of (e) is quantitative PCR. In alternative embodiments, embodiments, the amplification reaction of (e) is rolling circle amplification (RCA) or loop-mediated isothermal amplification (LAMP). In some embodiments, the Tm of the oligonucleotide is in the range of 65° C. to 95° C.

### BRIEF DESCRIPTION OF THE DRAWINGS

FIG. 1A-B: Overview of Identity Preserving Sample Multiplexing (IPSM) workflow. (A) Non-enzymatic barcoding, pooling and concurrent viral isolation from pooled patient cohorts. (B) Enzymatic screening of positive cohorts illustrated by quantitative polymerase chain reaction (qPCR) and sequencing-based quantification of patient viral load.

FIG. 2A-B: (A) Hybrid DNA and RNA construct to measure ligation sensitivity and specificity. The ligation junction mismatch substitutes an A for the complementary T. DNA template (red/black fragment) and ligation product (blue/orange fragment). (B) Template DNA (red) and ligation product (blue) are quantified by qPCR with distinct primers that share a similar amplification efficiency (by construction, the ligation product primers are the reverse of the DNA template primers).

FIG. 3: Illustration of three collinear oligonucleotides that hybridize to viral RNA.

FIG. 4: TaqMan qPCR for 50M, 5M & 500K T7 amplified viral templates as well as no template (NT), human GM12878 purified RNA (GM), no ligase (NL), and no barcode (NB) negative controls (N.D.=not detected)

FIG. 5A-B: (A) IPSM assay for titrated abundance of viral templates yields an estimated 5-50 molecule LoD by digital qPCR (BioRad). (B) Digital qPCR readout of IPSM measurement for SeraCare SARS-CoV-2 positive (˜65 viral particles) and negative (no viral particles) controls.

FIG. 6A-B: (A) Thermodynamically favorable α-oligonucleotide post-annealing configurations. (B) Synthetic viral samples with either no template (ϕ), GM12878 purified negative control RNA (GM), or SARS-CoV-2 RNA (C) were pooled for 30 minutes at room temperature as shown. Crosstalk is measured by qPCR (linear scale) and reflects the relative abundance of barcodes for pooled negative samples (ϕ or GM) compared with positive viral RNA controls (C). Note that crosstalk is nearly zero, showing that barcodes present in negative control samples do not promiscuously label positive sample RNA during pooling and subsequent processing.

FIG. 7: Stoichiometric sequencing control with Internal Cohort Balancing (ICB)

FIG. 8A-C: Internal patient cohort balancing by asymmetric, RNase-H dependent PCR. (A) Ct values for qPCR amplification of IPSM ligation product applied across a 1000-fold viral RNA titration. (B) PCR for IPSM ligation products following Internal cohort balancing (ICB). (C) Dynamic range (maximum-minimum) for pre- and post-ICB.

FIG. 9A-B: Internal patient cohort balancing for viral dilution series with next-generation sequencing read-out. (A) Post-ICB sequencing reveals uniform barcode sampling across dilution series. (B) UMI encoded viral titer recovered as the barcode library complexity.

### DETAILED DESCRIPTION OF THE INVENTION

### I. Terminology

As used herein, the terms “a”, “an”, and “the” can refer to one or more unless specifically noted otherwise.

The terms “about” and “approximately” as used herein shall generally mean an acceptable degree of error for the quantity measured given the nature or precision of the measurements. For example, exemplary degrees of error for temperature may be less than 5%, e.g., 4%, 3%, 2%, 1%, or 0.5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.”. Numerical quantities given herein are approximate unless stated otherwise, meaning that the term “about” or “approximately” can be inferred when not expressly stated. When “about” is applied to the beginning of a numerical range, it applies to both ends of the range.

The term “low secondary structure” in a RNA virus target sequence, e.g., a SARS-CoV-2 target sequence, refers to a region that is not predicted to form a helix through intramolecular base pairing between RNA nucleotides in the SARS-CoV-2 RNA genome. SARS-CoV-2 RNA secondary structure has been described (see, e.g., Rangan & Das, RNA genome conservation and secondary structure in SARS-CoV-2 and SARS-related viruses. BioRxiv, 2020). RNA secondary structure can also can be predicted using software for numerous other RNA structure prediction models, e.g., RNAfold, RNAstructure, and RNAshapes, CONTRAfold, CentroidFold, ContextFold, pknotsRG, Probknot, Pknot, Knotty, MC-Fold, MC-Fold-DP, CycleFold, and EvoClustRNA, among others.

The term “collinear” in the context of “collinear” oligonucleotides refers to oligonucleotides that hybridize to adjacent sequences of a target nucleic acid, such that there are no unhybridized intervening bases of the target nucleic acid sequence between the adjacent oligonucleotides.

A “polynucleotide” or “nucleic acid” includes any form of RNA or DNA, including, for example, genomic DNA; complementary DNA (cDNA), and DNA molecules produced synthetically or by amplification. “Polynucleotides” include nucleic acids comprising non-standard bases. A polynucleotide in accordance with the disclosure will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Polynucleotides may be single-stranded, double-stranded, or partially double-stranded. An “oligonucleotide” as used herein is preferably DNA; and includes embodiments in which an oligonucleotide contains one or more modified nucleotides.

As used herein, the term “complementary” refers to the capacity for precise pairing between two nucleotides. I.e., if a nucleotide at a given position of a nucleic acid is capable of hydrogen bonding with a nucleotide of another nucleic acid, then the two nucleic acids are considered to be complementary to one another at that position. A “complement” may be an exactly or partially complementary sequence. Two oligonucleotides are considered to have “complementary” sequences when there is sufficient complementarity that the sequences hybridize (forming a partially double stranded region) under assay conditions.

The terms “anneal”, “hybridize” or “bind,” in reference to two polynucleotide sequences, segments or strands, are used interchangeably and have the usual meaning in the art. Two complementary sequences (e.g., DNA and/or RNA) anneal or hybridize by forming hydrogen bonds with complementary bases to produce a double-stranded polynucleotide or a double-stranded region of a polynucleotide.

As used herein, “amplification” of a nucleic acid sequence has its usual meaning, and refers to in vitro techniques for enzymatically increasing the number of copies of a target sequence. Amplification methods include both asymmetric methods (in which the predominant product is single-stranded) and conventional methods (in which the predominant product is double-stranded).

As will be understood from context, every description of a method step or of an interaction of a reagent with SARS-CoV-2 RNA in a patient sample or pooled sample contemplates that the same steps or activities may be carried out in samples comprising SARS-CoV-2 (positive samples) and in samples that do not comprise comprising SARS-CoV-2 (negative samples). For example, the step of “ligating the three oligonucleotides hybridized to a SARS-CoV-2 nucleic acid” contemplates that ligase and oligonucleotides will be added to a negative pool, which will be maintained under ligation conditions, even though the oligonucleotides are not ligated together in a pool free from viral RNA.

### II. Introduction

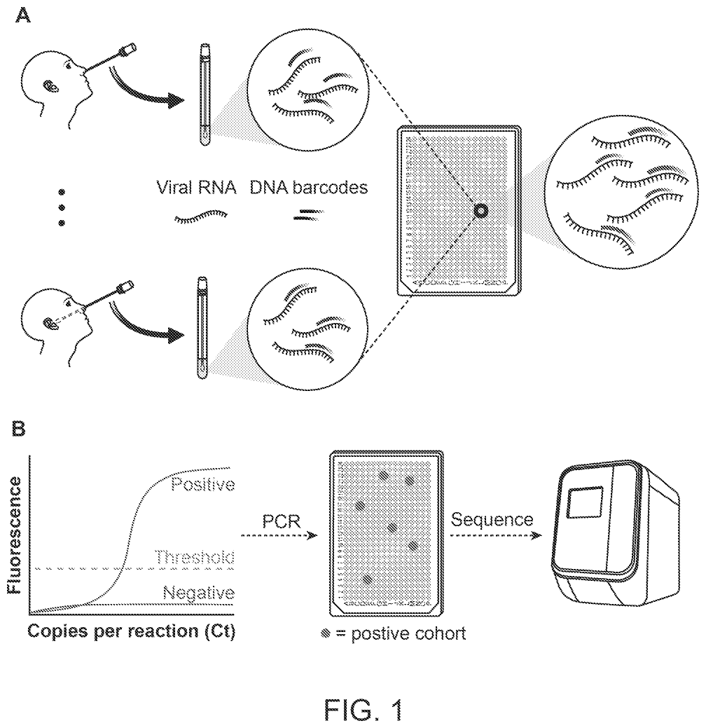

The IPSM technology described herein eliminates the retesting bottleneck of conventional pooling by individually labeling samples with patient-specific barcodes before pooling to preserve patient identities during pooled viral purification and enzymatic sample processing. This provides the ability to perform concurrent viral isolation, purification and enzymatic processing of 100-1000 patients per cohort, rapid screening of positive cohorts, and quantification of individual patient viral titers by massively-parallel barcode sequencing. A schematic of the method is provided in FIG. 1. Patients within negative cohorts can be cleared quickly, e.g., within two hours, while positive patients within positive cohorts are subsequently identified by barcode sequencing, again within a short period of time, e.g., 4 hours. The IPSM framework thus maintains analytic performance, while scaling testing throughput and reducing per-sample costs by over 10-fold.

Although the invention is largely described in the context of SARS-CoV-2 infection, the methods described herein can be employed for rapid screening for other viral infections, including other coronaviruses, such as SARS-CoV, MERS-CoV, or any other single-stranded RNA (ssRNA) virus. Further, the methodology can also be employed for rapid screening for single-stranded DNA (ssDNA) virus infections. Accordingly, the steps of the methods described herein, can be applied to detect other ssRNA or ssDNA viruses.

The patient screening methods of the present disclosure employ sequence-based barcodes, which provide trackable patient identifiers for SARS-CoV-2 sequences, if present, from a test sample obtained from a patient, thus allowing transcripts from pooled patient samples to be sequenced simultaneously in a single massively parallel sequencing pool without loss of the ability to trace the patient sample from which transcripts originated.

The present disclosure thus provides a method of rapidly identifying SARS-CoV-2-positive patients by incubating (i) a nucleic acid preparation from a patient with (ii) one, two, or three or more oligonucleotides that hybridize to target regions of SARS-CoV-2 RNA. One of the oligonucleotides comprises a patient-specific identification region, i.e., barcode. The oligonucleotides are incubated with the patient nucleic acid sample under conditions in which oligonucleotides can anneal to viral nucleic acids, if present in the sample. Following incubation, the patient sample is pooled with nucleic acid samples from other patients, e.g., from 10-100 different patients, that are similarly processed, but where the oligonucleotide(s) incubated with nucleic acid samples from different patient comprises different patient-specific identifying sequences.

Hybridized complexes comprising collinear oligonucleotides hybridized to SARS-CoV-2 RNA genome are isolated following pooling of nucleic acid samples; and, in instances in which two or more oligonucleotides are employed, ligated by RNA-splinted DNA ligation to ligate the oligonucleotides to provide a single oligonucleotide molecule comprising the patient-specific barcode hybridized to the SARS-CoV-2 nucleic acid. In embodiments in which a single oligonucleotide comprising a patient identifier sequence is hybridized to SARS-CoV-2 RNA instead of collinear oligonucleotides, a reverse transcriptase is employed to extend the hybridized oligonucleotide following pooling and isolation of hybridized oligonucleotide-SARS-CoV-2 complexes.

An amplification reaction, e.g., a quantitative PCR using SARS-CoV-2 primers, is then performed on a portion of the pool comprising the nucleic acids from different patients to determine whether the pool is positive or negative for the presence of SARS-CoV-2 polynucleotide sequences.

Positive pools are further processed for sequencing to balance the sequencing library so that SARS-CoV-2 sequences from patients having a high SARS-CoV-2 viral titer do not dominate the sequencing library and prevent identification of SARS-CoV-2 sequences from other patients who may have low viral SARS-CoV-2 titers. This procedure employs an asymmetric RNaseH-dependent PCR reaction to generate the balanced cohort sequencing library of nucleic acid molecules. For the asymmetric PCR, each patient-specific primer that targets the corresponding patient identifier barcode, is supplied in a common limiting concentration during PCR amplification. During this asymmetric PCR, each patient sub-library transitions from exponential to linear amplification once the patient-specific primer is consumed. The number of double stranded ligation products generated by this asymmetric PCR will then be narrowly distributed across all patients in the cohort. The library is then sequenced to determine the patient barcode sequences, thereby identifying patients that are positive for SARS-CoV-2.

### III. Oligonucleotides that Target SARS-CoV-2

In some embodiments, oligonucleotides for hybridization to SARS-CoV-2 RNA sequences are designed to target regions of the SARS-CoV-2 genome that have low secondary structure. In some embodiments, such oligonucleotides have a GC content of about 45% to about 55%. The oligonucleotides are thus designed to be stably bound to target during manipulations subsequent to annealing. One of skill understands how to work at temperatures that don't disrupt the duplex. Generally, SAR-CoV-2 binding region of an oligonucleotide provided herein can range in size from 15 to 50 nucleotides, although in some embodiments, the binding region may be longer. In some embodiments, the SAR-CoV-2 binding region is from 25 to 35 nucleotide in length. In some embodiments, the binding region is 25, 26, 27, 28, 29, 30, 31, 32, 33, 34, or 35 nucleotides in length.

In embodiments employing multiple collinear oligonucleotides to target SAR-CoV-2, the Tm of an oligonucleotide that does not comprise a patient identifier sequence, e.g., an oligonucleotide that binds to a SARS-CoV-2 target region positioned between the sequences to which flanking oligonucleotides bind, has a Tm that is about the temperature of the ligation reaction in which collinear oligonucleotides are joined, or higher. For example, ligation reactions can be performed at room temperature or higher. Thus, in some embodiments, the oligonucleotide-viral RNA duplex may have a Tm of at least about 22° C. In some embodiments, the Tm is at least 10° C. higher, or at least 20° C. greater than the temperature at which the ligation reaction is performed. In some embodiments, the oligonucleotides are designed to have a Tm of at least 50° C. or at least 55° C. or at least 60° C. In other embodiments, the Tm is at least 65° C. In some embodiments, the Tm is at least 70° C. In some embodiments, the Tm is at least 75° C. In some embodiments, the Tm is at least 80° C. or at least 85° C. In some embodiments, suitable oligonucleotides have a Tm in the range of about 45° C. to about 95° C. In some embodiments, the Tm is in the range of about 50° C. to about 95° C. In some embodiments, the Tm is in the range of about 55° C. to about 95° C. In some embodiments, the Tm is in the range of about 60° C. to about 95° C. In some embodiments, the Tm is in the range of about 65° C. to about 90° C. Tm can be calculated using known methods, for example, the www http address idtdna.com/pages/tools/oligoanalyzer.

An oligonucleotide that comprises a patient identifier sequence is generally designed to have a Tm that is at least about 20° C. above the temperature at which collinear oligonucleotides are ligated or reverse transcription is conducted. Thus, for example, in embodiments employing collinear oligonucleotides, the Tm of an oligonucleotide that comprises the patient identifier region is generally designed to be above about 42° C., i.e., 20° C. above a room temperature ligation reaction. In embodiments in which a single oligonucleotide comprising the patient identifier region is extended by reverse transcriptase, the oligonucleotide may have a Tm of least about 62° C., i.e., 20° C. above a reverse transcription reaction. Accordingly, in some embodiments, the Tm is at least about 45° C. In some embodiments, the Tm is at least 50° C. or at least 55° C. or at least 60° C. In other embodiments, the Tm at least 65° C. In some embodiments, the Tm is at least 70° C. In some embodiments, the Tm is at least 75° C. In some embodiments, the Tm is at least 80° C. or at least 85° C. In some embodiments, suitable oligonucleotides have a Tm in the range of about C to about 95° C. In some embodiments, the Tm is in the range of about 50° C. to about C. In some embodiments, the Tm is in the range of about 55° C. to about 95° C. In some embodiments, the Tm is in the range of about 60° C. to about 95° C. In some embodiments, the Tm is in the range of about 65° C. to about 90° C.

In embodiment in which multiple collinear oligonucleotides are employed, the Tms of the individual oligonucleotides may differ. In some embodiments, the Tms are within 5° C. or 10° C. of one another. In some embodiments, the Tms are the same.

In some approaches, a target hybridization region is a region that will anneal to oligonucleotide(s) having a GC content of about 45% to about 55%. In a preferred embodiment, the target hybridization region is a region of low secondary structure in the SARS-CoV-2 RNA sequence. For example, in some embodiments in which multiple, e.g., three, collinear oligonucleotides are used, the oligonucleotide that binds to the region between the 5′-most and 3-most oligonucleotides may bind at a region starting at position 28448 within the N gene of SARS-CoV-2, as defined using the MT007544.1 genome build (NCBI, Severe acute respiratory syndrome coronavirus 2 isolate Australia/VIC01/2020, complete genome).

In some embodiments, the oligonucleotide(s) comprise one or more modified nucleotides. Any suitable modified nucleotide may be included, but in some embodiments, the modification includes a Tm-enhancing modification, that is, a modification that increases Tm relative to an oligonucleotide that has the same sequence, but does not include the modification. Such Tm-enhancing modifications include, for example, a modified 5-methyl deoxycytidine (5-methyl-dc); 2,6-diaminopurine; a locked nucleic acid (LNA); a bridged nucleic acid (also referred to as a bicyclic nucleic acid or BNA); a tricyclic nucleic acid; a peptide nucleic acid (PNA); a CS-modified pyrimidine base; a propynyl pyrimidine; a morpholino; a phosphoramidite; or a 5′-Pyrene cap. In embodiments in which multiple oligonucleotides are employed for annealing and subsequent ligations, each of the oligonucleotides typically comprises the same type of modified nucleotides to increase Tm.

The same oligonucleotide design considerations, such as Tm, GC content, and length of SARS-CoV-2 binding region detailed above are employed for embodiments in which one oligonucleotide is to be annealed to target viral RNA and extended by reverse transcriptase after annealing and pooling of the patient sample with other samples.

In some embodiments at least two, and preferably at least three, oligonucleotides are annealed to SARS-CoV-2 and subsequently ligated to each other. In such embodiments, the SARS-CoV-2 binding region of each of the oligonucleotides may be of the same length. Alternatively, the SARS-CoV-2 binding region of each oligonucleotide may differ in length. For example, in some embodiments, the binding regions may differ in length by 1-5 nucleotides, or by 1-10 nucleotides.

Embodiments in which collinear oligonucleotides are annealed to viral nucleic acids and joined by ligation typically employ three oligonucleotides. However, in some embodiments, more than three oligonucleotides, e.g., 4 or 5, may be used to increase specificity.

### IV. Patient Identifier Sequences

Identification of a patient that is infected with SARS-CoV-2 is achieved through the use of patient-specific identifier sequences, i.e., barcodes, incorporated at the 5′ or 3′ end of at least one of the oligonucleotides that is incubated with a patient sample for annealing to SARS-CoV-2 RNA, when present in the sample. For embodiments employing one oligonucleotide in which an oligonucleotide annealed to viral target RNA is extended using RT, the barcode sequence is present at the 5′ end of the oligonucleotide. For embodiments in which multiple oligonucleotides are annealed to viral target RNA and ligated, the barcode sequence may be present at the 3′ end of the oligonucleotide that targets the region that is the farthest upstream (i.e., 5′), relative to the target regions of the other oligonucleotide(s) (also referred to herein as “5′-most” oligonucleotide. Thus, when the multiple oligonucleotides are ligated to one another, the barcode is at the 3′ end of the ligated product. Alternatively, the barcode sequence may be present at the 5′ end of the oligonucleotide that targets the region that is farthest downstream (i.e., 3′) relative to the target region of the other oligonucleotide(s) (also referred to herein as “3′-most”). Accordingly, when the multiple oligonucleotides are ligated to one another, the barcode is at the 5′ end of the ligated product. In some embodiments, the barcode sequence may be included at both the 3′ end of the oligonucleotide that hybridizes to the target at the position farthest upstream, and the 5′ end of the oligonucleotide that hybridizes to the target at the position farthest downstream. The resulted ligated product when the oligonucleotides are ligated to one another will then contain the patient-specific identifying region at both the 3′ and 5′ ends.

The patient-specific identifying regions are typically the same size relative to one another. In some embodiments, the size may be anywhere from 15-25 nucleotides in length, for example, 15, 16, 17, 18, 19, or 20 nucleotides in size. In some embodiments, the barcode region is 16 nucleotides in length.

The barcode sequences are designed to result in one or more base-pair mismatches if the barcode hybridizes to any primer (for the RNase H asymmetric extension, as detailed below) other than the primer specific for the particular patient-specific barcode. In some embodiments, the barcode sequences are selected for a Hamming distance of 1, 2, 3, 4, 5, or 6, or more, nucleotides up to the length of the barcode sequence. In some embodiments, the barcode sequences are selected for a Hamming distance of 4 nucleotides. Additional considerations in barcode design include the GC content (preferably about 50%) and incorporation of an RNA base for the corresponding primer used in the RNaseH-dependent PCR.

### V. Additional Sequence Elements

An oligonucleotide that anneals to the viral nucleic acid target may also comprise additional sequences, such as a unique molecular identifier that identifies sequences that are amplified from the same initial template molecule; and a universal amplification sequence, i.e., a primer binding site for a universal primer.

In some embodiments, the oligonucleotide is designed to contain a sequence that forms a hairpin. For example, an oligonucleotide that comprises the barcode at the 3′ end and hybridizes to the 5′-most target sequence in the viral nucleic acid may be designed such that the first few, e.g., 4-12, non-complementary bases are reverse complementary to the initial (complementary) bases of the oligonucleotide, forming a stem-loop structure in the absence of viral templates. As the temperature is decreased during annealing, the hairpin region adopts one of two thermodynamically favorable configurations: it is either specifically annealed to the viral RNA template, or collapsed as a sequestered hairpin, which can't anneal to viral RNA after pooling with other patient samples.

Oligonucleotides for annealing to target viral nucleic acids as described herein are also often attached to a molecule that allows for easy purification. Thus, for example, an oligonucleotide may be biotinylated, e.g., at the 5′ end. Examples of other purification moieties molecules include a hapten, a ligand that binds to a cognate binding partner, or an alternative purification tag.

### VI. Incubation of Oligonucleotides with Patient Nucleic Acids

Viral RNA is extracted from a sample obtained from a patient to be evaluated for SARS-CoV-2 infection. The sample may be from a throat swab, a nasopharyngeal swab, sputum or tracheal aspirate, or any other sample that may contain viral nucleic acids. At least one oligonucleotide as described above, which comprises a patient-specific identifier sequence, is then incubated with the nucleic acids extracted from the sample under conditions suitable for annealing, i.e., conditions in which the oligonucleotide will anneal to target SARS-CoV-2 sequences. In one approach, the samples are heated to a temperature above the Tm of the oligonucleotide, and then cooled, e.g., allowed to cool to room temperature, so that the oligonucleotide anneals to the target sequence, if present in the sample, and provides a stable hybridization complex in which oligonucleotides hybridized to the viral nucleic acid remains hybridized when pooled with other samples and throughout subsequent manipulations.

RNA-containing samples obtained from each of a plurality of patients are separately incubated with one or more oligonucleotides. As explained above, the patient-specific identifying sequence for each patient differs in sequence from the patient-identifying sequences for other patients. The barcode-comprising oligonucleotides in the separate incubations thus contain distinct barcodes for each patient. Samples can be separately incubated in droplets, microfluidic devices, wells, tubes, or any other compartments in which each patient samples is in a separate compartment.

Following the annealing step, the patient nucleic acid preparation containing the oligonucleotides (hybridized to SARS-CoV-2 viral RNA, if it is present) is pooled with the nucleic acid preparations from other patients that were similarly processed, i.e., incubated with an oligonucleotide comprising a barcode region that is specific for each patient under conditions in which the oligonucleotide will anneal to the target viral sequence if it is present in the sample. Hybridization complexes are then purified from the pool, e.g., via a biotin tag.

In some embodiments, in order to mitigate potential binding of oligonucleotides that comprise other patient identifier sequences the oligonucleotide comprising the patient-specific barcode is added in significant molar excess, e.g., 2-fold or 5-10-fold, of target viral RNA in the annealing incubation to block specific binding of alternate barcodes after pooling. In some embodiments, a single-stranded DNA nuclease, e.g., a 5′ exonuclease, is added after annealing, but prior to pooling, to remove free, i.e., unannealed, oligonucleotides that may otherwise anneal at room temperature.

### VII. Ligation/RT Reaction

In embodiments in which at least two, and preferably at least three, collinear oligonucleotides are annealed to the target viral nucleic acid and ligated to one another via RNA-splinted DNA ligation. An example of an RNA-splinted DNA ligase is Chlorella virus DNA ligase (PBCV-1 DNA ligase) (see, e.g., Lohman et al., Nucleic Acids Res. 42:1831-1844, 2014) or an analog or homolog thereof. PBCV-1 DNA ligase ligates adjacent, single-stranded DNA splinted by a complementary RNA strand. In preferred embodiments, at least three collinear oligonucleotides annealed to target viral RNA are ligated to provide at least two ligations, which can reduce or eliminate non-specific ligation events.

In embodiments in which one oligonucleotide is used to incubate with patient nucleic samples in the annealing step, the oligonucleotide annealed to the viral target region is extended using reverse transcriptase.

### VIII. Determination of Positive Pools

Following ligation, or reverse transcription, a portion of the pooled nucleic acid sample is then amplified to determine whether or not a pool contains SARS-CoV-2 sequences. Any type of amplification reactions can be used. In some embodiments, qPCR is performed using SARS-CoV-2 specific primers to amplify viral nucleic acids.

Alternative amplification reactions to determine positive pools include T7 amplification, rolling circle amplification (RCA), loop-mediated isothermal amplification (LAMP) or any other suitable amplification reaction. For example, LAMP or RCA amplification reactions can be employed to generate a fluorescently amplified product that can be quantified.

Pools that are determined to be negative are not analyzed further. Positive pools are further processed in preparation for sequencing.

### IX. RNase H-Dependent PCR

A positive pool is processed to balance the library to provide a balanced cohort sequencing library such that SARS-CoV-2 sequences from patients having a high SARS-CoV-2 viral titer do not dominate the sequencing library and prevent identification of SARS-CoV-2 sequences from other patients who may have very low viral SARS-CoV-2 titers. This procedure employs an asymmetric RNase H-dependent PCR reaction.

For the asymmetric PCR, each patient-specific primer that targets the corresponding patient identifier barcode, is supplied in a common limiting concentration during PCR amplification. During this asymmetric PCR, each patient sub-library transitions from exponential to linear amplification once the patient-specific primer is consumed. The number of double stranded ligation products generated by this asymmetric PCR will then be narrowly distributed across all patients in the cohort. The library is then sequenced to determine the patient barcode sequences, thereby identifying patients that are positive for SARS-CoV-2.

RNase-dependent PCR reactions are known (see e.g., Dobsy et al, BMC Biotechnology, 11:80. 2011). The reaction employs a cleavable RNA base in a PCR primer to increase specificity. In some embodiments, the RNA base is incorporated at or near, e.g., within 1, 2, 3, 4, 5, 6, or 7 nucleotides of the 3′ end of the primer. The following examples of primer sequences are provided to illustrate the primer sequences that hybridize to the patient-specific identifier regions:

(SEQ ID NO: 1)
AGAGCACTAGTCrAACGAA/3SpC3/
(SEQ ID NO: 2)
TGCCTTGATCGArACGATG/3SpC3/
(SEQ ID NO: 3)
CTACTCAGTCAGrAGTAGA/3SpC3/
(SEQ ID NO: 4)
TCGTCTGACTCTrATGTGT/3SpC3/
(SEQ ID NO: 5)
GAACATACGGGArCACCAT/3SpC3/
(SEQ ID NO: 6)
CCTATGACTCTGrCCAACT/3SpC3/
(SEQ ID NO: 7)
GAGCGCAATACTrCGATCG/3SpC3/
(SEQ ID NO: 8)
AACAAGGCGTACrCTAGCG/3SpC3/
(SEQ ID NO: 9)
ATGTCGTGGTTGrGATCGA/3SpC3/
(SEQ ID NO: 10)
TTGCCGAGTGTrGCTCTC/3SpC3/.

The 6th from the last base is the cleavable RNA base, the terminal 3′ base is a mismatch and each primer is blocked at the 3′ end with a spacer.

The above procedure provides a balanced cohort library from a positive pool. The library is then processed for sequencing using high throughput sequencing methodology.

### X. Illustrative Protocol

An illustrative protocol employing three collinear oligonucleotides for annealing to SARS-CoV-2 RNA, if present in a patient sample; and reagents for performing the method are provided below. Sequences referred to in the protocol are provided at the end. One of skill understands that variations will be recognized by those of skill in the art.
1. Rinse patient swab in 40 μl in Qiagen viral RNA lysis buffer (Qiagen, #52904) supplemented with 100 mM NaCl and a patient-specific, barcoding oligonucleotide (α,β,γ)-triple, sequence provided at the end of this example.
2. Anneal 20 μl aliquots of each patient sample by incubating at 94° C. for 30 seconds and slowly decreasing the temperature from 94° C. to 42° C. at 2° C. per minute
3. Pool 10 μl of each sample in two non-overlapping patient pools. Add H₂O to adjust total pool volume to 140 μl.
4. Purify pooled samples using the Qiagen viral RNA mini kit (Qiagen, #52904) according to the manufacture's protocol and elute in 20 μl.
5. Perform 50 μl Taqman qPCR (50 cycles) on a 384 well plates using IDT PrimeTime Gene Expression Master Mix (IDT, 1055772) according the manufacture's protocol with the patient barcode (PB) primer pool and P7 as forward and reverse primer pairs, and T1 as the Taqman probe.
6. Report negative results for all patient samples contained in negative sample pools with Ct values above 45 cycles.
7. Purify positive pools with 2X (100 μl) SPRI (Beckman Coulter, A63880) according to the manufactures protocol.
8. Dilute positive pools from step 7 1000-fold and perform assymetric RNaseH-dependent PCR for 20 cycles with R1PB primer pool at 0.09 μM and P7 at 0.9 μM. The R1PB primer pool is the pool of RNase-H-dependent primers with a TrueSeq R1 sequence at the 5′ end for hybridization to the i5.idx primers for pool indexing.
9. Purify asymmetric PCR product with 2X (100 μl) SPRI (Beckman Coulter, A63880) according to the manufacture's protocol and elute in 20 ul.
10. Add an i5 Illumina adapter i5.idx with a unique index using 2 μl of each ICB pool from step 9 (5 cycle, 50 μl PCR with reverse primer P7). Purify PCR product with 2X (100 μl) SPRI (Beckman Coulter, A63880) according to the manufacture's protocol and elute in 20 μl.
11. Quantify each pooled library by Qubit (ThermFisher Scientific, #Q33327) and prepare a 4 nM sequencing library with equal contribution for each cohort.
12. Sequence library using an Illumina sequencer (Read 1: 28 bp, Index 2 read: 8 bp). Collect at least 10,000 reads per positive patient sample.
13. Estimate barcode abundance for each patient sample by counting unique molecular identifiers (UMIs) and extrapolating patient barcode diversity under a Poisson sampling model.
14. Use negative samples (including both contrived as well as patient samples in negative cohorts) to construct a background distribution (empirical null model) of non-specific reads and assign a p-value to each sample.

Patients for which both paired samples admit a p-value less (greater) than a selected false positive rate (FPR) are reported as positive (negative). Patients with discordant p-values (one above and one below the FPR threshold) are considered indeterminate and will be re-processed using the remaining 20 μl of barcoded sample from step 2.

Sequences referred to in protocol example:
P7:
(SEQ ID NO: 11)
CAAGCAGAAGACGGCATACGAGAT
T1:
(SEQ ID NO: 12)
/56-FAM/TGGTCATCTGGACTGCTATTGGTGT/3BHQ_1/
PB primer pool (patient barcodes):
(SEQ ID NO: 13)
AGAGCACTAGTCAACGAT
(SEQ ID NO: 14)
TGCCTTGATCGAACGATC
(SEQ ID NO: 15)
CTACTCAGTCAGAGTAGT
(SEQ ID NO: 16)
TCGTCTGACTCTATGTGA
(SEQ ID NO: 17)
GAACATACGGGACACCAA
(SEQ ID NO: 18)
CCTATGACTCTGCCAACA
(SEQ ID NO: 19)
GAGCGCAATACTCGATCC
(SEQ ID NO: 20)
AACAAGGCGTACCTAGCC
(SEQ ID NO: 21)
ATGTCGTGGTTGGATCGT
(SEQ ID NO: 22)
ATTGCCGAGTGTGCTCTG
R1PB primer pool
(SEQ ID NO: 23)
ACACTCTTTCCCTACACGACGCTCTTCCGATCTGCAGAGTAGAGCACTAGTCrAAC
GAT/3SpC3/
(SEQ ID NO: 24)
ACACTCTTTCCCTACACGACGCTCTTCCGATCTGCAGAGTTGCCTTGATCGArACG
ATC/3SpC3/
(SEQ ID NO: 25)
ACACTCTTTCCCTACACGACGCTCTTCCGATCTGCAGAGTCTACTCAGTCAGrAGT
AGT/3SpC3/
(SEQ ID NO: 26)
ACACTCTTTCCCTACACGACGCTCTTCCGATCTGCAGAGTTCGTCTGACTCTrATG
TGA/3SpC3/
(SEQ ID NO: 27)
ACACTCTTTCCCTACACGACGCTCTTCCGATCTGCAGAGTGAACATACGGGArCA
CCAA/3SpC3/
(SEQ ID NO: 28)
ACACTCTTTCCCTACACGACGCTCTTCCGATCTGCAGAGTCCTATGACTCTGrCCA
ACA/3SpC3/
(SEQ ID NO: 29)
ACACTCTTTCCCTACACGACGCTCTTCCGATCTGCAGAGTGAGCGCAATACTICGA
TCC/3SpC3/
(SEQ ID NO: 30)
ACACTCTTTCCCTACACGACGCTCTTCCGATCTGCAGAGTAACAAGGCGTACICTA
GCC/3SpC3/
(SEQ ID NO: 31)
ACACTCTTTCCCTACACGACGCTCTTCCGATCTGCAGAGTATGTCGTGGTTGrGAT
CGT/3SpC3/
(SEQ ID NO: 32)
ACACTCTTTCCCTACACGACGCTCTTCCGATCTGCAGAGTATTGCCGAGTGTrGCT
CTG/3SpC3/
I5.idx
(SEQ ID NO: 33)
AATGATACGGCGACCACCGA[8 bp pool barcode]ACACTCTTTCCCTACACGAC
GCTCTTCCGATC
α-oligonucleotide:
(SEQ ID NO: 34)
/5Phos/TCGAGGGAATTTAAGGTCTTCCTTGCCATGTCGANNNNNNNNNN[barcode
from PB primer pool]
ß-oligonucleotide:
(SEQ ID NO: 35)
CAAGCAGAAGACGGCATACGAGATTCGGTAGTAGCCAATTTGGTCATCTGGAC
T
γ-oligonucleotide:
(SEQ ID NO: 36)
/5Phos/GCTATTGGTGTTAATTGGAACGCCTTGTCC

It is understood that the examples and embodiments described throughout the specification are for illustrative purposes only and that various modifications or changes in light thereof will be suggested to persons skilled in the art and are to be included within the spirit and purview of this application and scope of the appended claims.

### EXAMPLES

The examples provide data illustrating aspect of Identity Preserving Sample Multiplexing (IPSM) technology, which preserves the identity of patient samples by non-enzymatically barcoding each patient virus sample prior to pooling, purification, concurrent enzymatic processing and patient barcode sequencing. In particular, the examples illustrate high sensitivity sample barcoding (the limit of detection is currently less than 50 molecules and on track for single-digit sensitivity), low levels of crosstalk between pooled patient samples (fewer than 1 in 1,000,000 barcodes misrepresent the patient origin), and efficient, massively parallel sequencing of patient barcodes using a pool balancing technique termed internal cohort balancing (ICB).

### Example 1. Non-Enzymatic Sample Barcoding

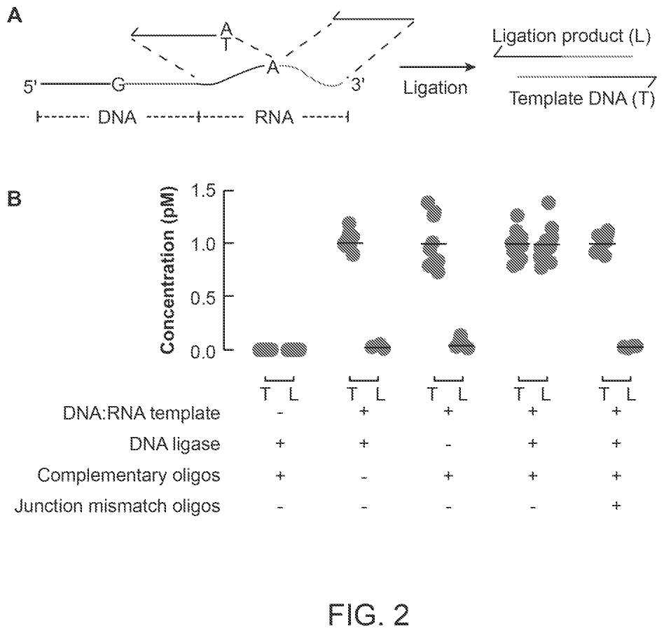

Patient samples were barcoded by annealing high melting-temperature DNA oligonucleotides to lysed viral RNA. Annealing was highly efficient across a broad range of lysis conditions, and barcodes remained stably bound during subsequent purification and ligation reactions, as these procedures are executed at room temperature, well below the melting temperature of the barcoding oligonucleotides. To test the specificity and sensitivity of both annealing and RNA-splinted DNA ligation, we synthesized a hybrid oligonucleotide sequence comprising a 52 base pair RNA sequence that is the complement of a covalently linked DNA sequence (FIG. 2A). We then annealed and ligated (SplintR® ligase, New England Biolabs) a pair of complementary oligonucleotides in complex with the RNA component under a variety of control conditions (FIG. 2B). These measurements showed that the ligation product had nearly identical abundance to the template (99.8% ligation efficiency) and, furthermore, that a single base pair mismatch at the ligation junction nearly ablates ligation (FIG. 2B). Collectively, these data showed that RNA-splinted DNA ligation is a highly sensitive, specific, and quantitative readout for RNA.

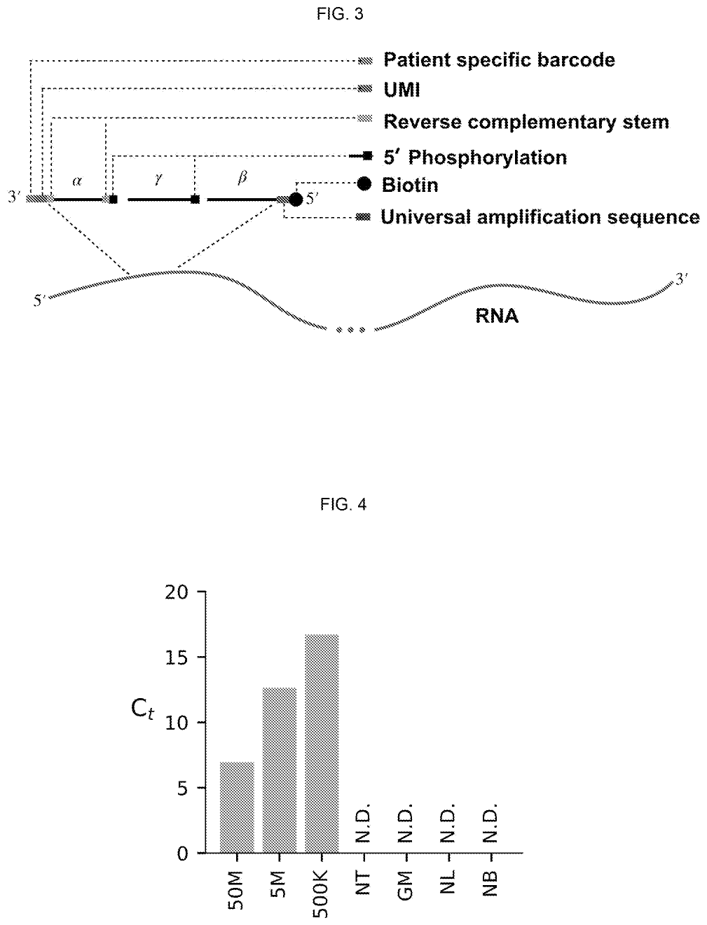

Although the DNA splinted ligase SplintR is highly specific to base pair mutations at the ligation junction, short regions of perfect complementarity near the ligation junction may exist among sequences present in co-isolated host RNA. In order to reduce or eliminate these non-specific ligation events, we adopted a dual ligation scheme in which three collinear oligonucleotides were annealed and subsequently ligated following purification (FIG. 3). Each sample was independently pooled in multiple, non-overlapping patient cohorts (one barcode locus per cohort). Target sites within the SARS-CoV-2 genome were chosen with low secondary structure (Rangan & Das, RNA genome conservation and secondary structure in SARS-CoV-2 and SARS-related viruses. BioRxiv, 2020) and similar GC content (45-55%) across the barcoding oligonucleotides. To test the specify and sensitivity of the proposed assay we quantitatively measured (TaqMan qPCR) the abundance of ligation products across a range of input templates generated by T7 amplification of a SARS-CoV-2 viral fragment containing the barcode target region (FIG. 4). We observed an expected scaling pattern for these positive samples, while samples lacking template (NT), ligase (NL), or (α, β, γ) barcodes (NB) yielded no signal/product after 80 cycles of PCR amplification. Similarly, samples containing GM12878 purified RNA (GM) yielded no detectable ligation product. Collectively these data establish highly specific and sensitive viral RNA detection by the proposed ligation-mediated qPCR.

### Example 2. Limit of Detection (LoD)

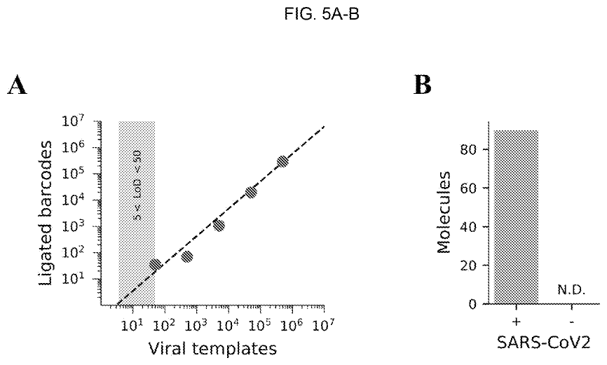

To estimate the limit of detection, we synthesized a 252 bp RNA fragment containing the barcode target region, and performed the detection assay across a titration of more than 100,000 to fewer than 50 molecules. We then used digital droplet TaqMan PCR (ddPCR, BioRad) to directly count the number of ligation events in each sample. These data establish a current limit of detection between 5 molecules (the detection limit of the ddPCR platform) and 50 molecules (the lowest abundance template tested to date, FIG. 5A). In addition, we independently confirmed low abundance detection by isolating viral RNA from a control SARS-CoV-2 virus (AccuPlex SARS-CoV-2 Reference Material Kit, SeraCare) and recording a similar number of ligation events to the number of input viral particles (FIG. 5B). Given the observed efficiency of SplintR ligation (FIG. 2), optimization of the TaqMan qPCR readout should show a single-digit detection limit.

### Example 3. Minimizing Cross-Talk in Pooled Samples

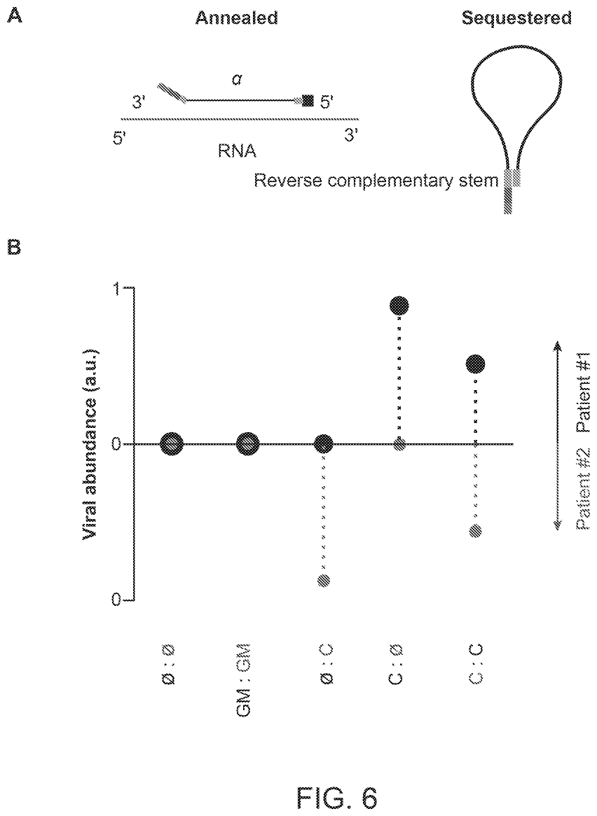

In this example, we opted to detect RNA by ligation because of a unique property of how the barcode is oriented: the barcoding oligonucleotide (a, see FIG. 3) is reverse complementary to the sense viral sequence, so it cannot be spuriously linked to viral RNA from other patients during post-ligation PCR amplification. Because reverse-sense sample barcoding eliminates PCR-mediated crosstalk between samples, the only mechanism available for crosstalk is direct cross-annealing after sample pooling. This form of potential crosstalk can be mitigated in four different ways. First, patient barcodes are added in significant molar excess of target viral RNA and block specific binding of alternate barcodes after pooling. Second, in order to remove free barcodes that may otherwise anneal at room temperature, a 5′ exonuclease is added after annealing, but prior to pooling. Third, the a-oligonucleotide is designed as a hairpin such that its first few non-complementary bases are reverse complementary to the initial (complementary) bases of the oligonucleotide, forming a stem-loop structure in the absence of viral template (FIG. 3, FIG. 6A). As the temperature is decreased during annealing, α-oligonucleotides adopt one of two thermodynamically favorable configurations: they are either (1) specifically annealed to the viral RNA template, or (2) collapsed as a sequestered hairpin, incapable of annealing to RNA after pooling with other patient samples (FIG. 6A). Fourth, even if low levels of crosstalk persist, we can analytically model and compensate for this effect in a manner conceptually similar to flow cytometry spectral compensation. As shown in FIG. 6B, we directly measured sample crosstalk and observed fewer than 1 in 1,000,000 crosstalk events when the only mitigation is excess α-oligonucleotides.

### Example 4. Barcode Sequencing with Internal Cohort Balancing (ICB)

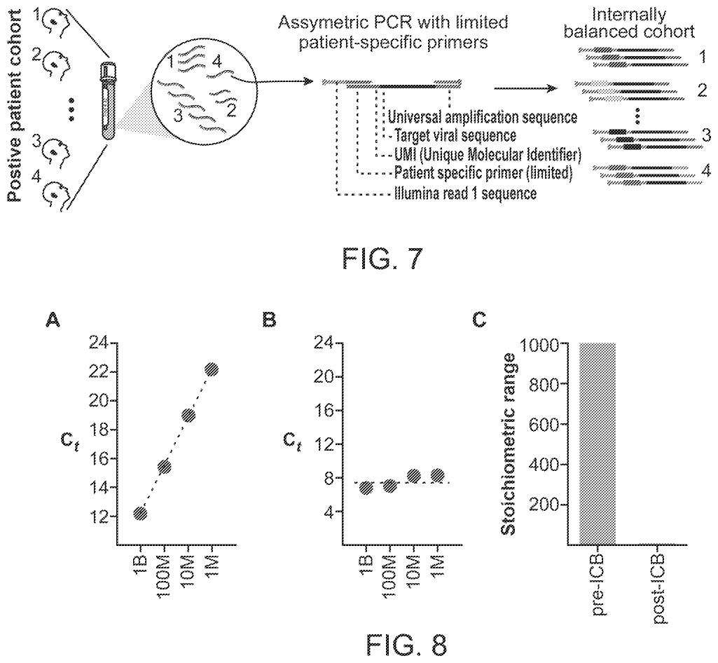

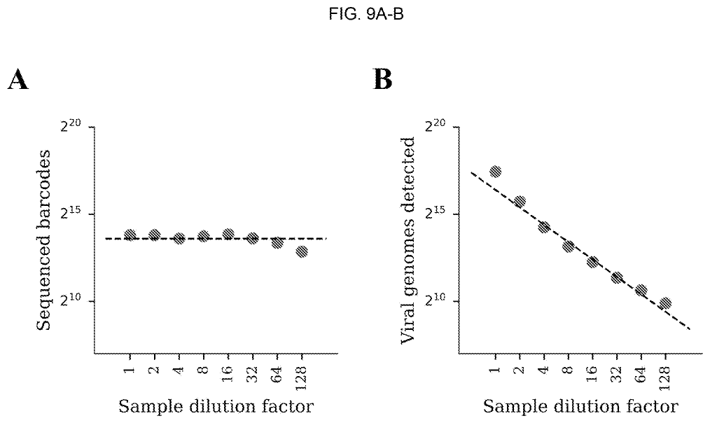

In order to measure the abundance of viral RNA for each sample within a pooled cohort by sequencing, a balanced library is constructed such that each patient sub-library is similarly represented. Otherwise low-titer positive samples will be obscured by over-sequenced patient samples with high viral load. While it is straightforward to balance reads across separate patient pools, building a library that balances reads within a patient pool is important, given that viral loads vary by many orders of magnitude among patients. To solve this problem, we developed a novel framework for internal cohort balancing (ICB) using asymmetric PCR (FIG. 7). The concept is to amplify ligation products from each patient using patient-specific primers that are supplied in a (common) limiting concentration during PCR amplification. During this asymmetric PCR, each patient sub-library transitions from exponential to linear amplification after the patient-specific primer is consumed. The number of double stranded ligation products generated by this asymmetric PCR will then be narrowly distributed across all patients in the cohort. To test the ICB concept, we performed asymmetric PCR across samples ranging from 1 million to 1 billion copies (FIG. 8A). We then quantified the relative abundance of each library and found that the initial 1000-fold stoichiometric range was reduced to less than 2-fold variation after ICB (FIGS. 8B-C). Note that, although the number of sequenced reads will be similar for each positive sample (for efficient sampling), the complexity of each patient library—representing the viral abundance—can be estimated from the diversity of unique molecular identifiers (UMIs) encoded within the barcoding α-oligonucleotide (FIG. 3). To test this unusual concept experimentally, we performed ICB on a dilution series of 8 IPSM samples where each sample contained half the number of viral genomes of the previous sample in the series. By construction, the viral load of these samples varied by more than 100-fold, yet ICB reduced the stoichiometric range of the sequenced barcodes to less than 2-fold (FIG. 9A). As predicted, we were then able to quantitatively recover the viral load of each sample by calculating the diversity of unique molecular identifiers encoded within the α-oligonucleotide (FIG. 9B). These experiments demonstrate that the ICB technology robustly encodes patient viral abundance independently of patient sub-library stoichiometry. This allows for efficient, uniform sampling of patient barcodes while quantitatively preserving the clinically relevant viral titer of each patient.

For the ICB approach to work at scale in a multiplexed, asymmetric PCR reaction (e.g., 100-1000 patients per cohort), it is important that each patient sub-library saturates independently. Consequently, we have employed RNaseH-dependent PCR to ensure that patient-specific primers do not cross-amplify mismatched targets. The degree to which barcodes are resolved by this mechanism will determine the maximum achievable patient cohort size. A distinct advantage of this “balance & sequence” approach over a naïve patient-specific qPCR readout is that even if the ICB PCR is not perfectly specific, crosstalk only effects the efficiency of the sequencing read-out (by introducing an imbalanced representation of samples) and does not result in wrongly diagnosed patients.

Examples 1-4 thus support that this method is robust and promises to dramatically reduce per-sample sequencing costs.

### Example 5. Illustrative Target Regions

In this example, a region of low secondary structure in the SARS-CoV-2 RNA that also provides for design of oligonucleotide having a GC content of about 45% to about 55% serves as the target hybridization region. For example, in some embodiments, oligonucleotides may bind at a region starting at position 28448 within the N gene of SARS-CoV-2 (e based on the MT007544.1 genome build (NCBI, Severe acute respiratory syndrome coronavirus 2 isolate Australia/VIC01/2020, complete genome). Examples of sequences of the SAR-CoV-2-targeting region of three collinear oligonucleotides designated as alpha, beta, or gamma as designated in FIG. 3: are:

SARS-CoV-2_N_28448_30 bp_alpha:
(SEQ ID NO: 37)
5′-TCGAGGGAATTTAAGGTCTTCCTTGCCATG-3′
SARS-CoV-2_N_28448_30 bp_gamma:
(SEQ ID NO: 38)
5′-GCTATTGGTGTTAATTGGAACGCCTTGTCC-3′
SARS-CoV-2 N_28448_30 bp_beta:
(SEQ ID NO: 39)
5′-TCGGTAGTAGCCAATTTGGTCATCTGGACT-3′.

An example of a complete alpha oligonucleotide sequence is: /5Phos/TCGAGGGAATTTAAGGTCTTCCTTGCCATGTCGANNNNNNNNNN (SEQ ID NO:40). The self-complementary sequences TCGA are shown in bold, as is the barcode, represented by “N”.

An example of a complete beta oligonucleotide sequence is:

(SEQ ID NO: 41)
5′CAAGCAGAAGAC
GGCATACGAGATTCGGTAGTAGCCAATTTGGTCATCTGGACT 3′.

The sequence shown in bold is a universal amplification sequence.

All publications, patents, and patent applications cited herein are hereby incorporated by reference with respect to the material for which they are expressly cited.

1. A method of rapid identification of SARS-CoV-2 positive patient(s) within a group comprising a plurality of patients in need of evaluation for SARS-CoV-infection comprising:
(a) separately incubating RNA-containing samples obtained from each of the plurality of patient with three or more collinear oligonucleotides,
wherein at least one of the three or more collinear oligonucleotides comprises a patient-specific identifying barcode sequence,
wherein the three or more collinear oligonucleotides have sequences complementary to a SARS-CoV-2 RNA target sequence, under conditions in which, if an RNA-containing sample comprises SARS-CoV-2 genomic RNA, an oligonucleotide-SARS-CoV-2 RNA complex is produced comprising the three or more collinear oligonucleotides hybridized at adjacent positions in the SARS-CoV-2 RNA target sequence,
thereby producing a plurality of incubated patient samples each of which comprises an oligonucleotide with a different patient-specific identifying barcode sequence, and
(b) pooling the plurality of incubated patient samples to produce a pooled sample;
(c) purifying oligonucleotide-SARS-CoV-2 complexes, when present, from the pooled sample;
(d) ligating the three or more collinear oligonucleotides hybridized to SARS-CoV-2 RNA in the oligonucleotide-SARS-CoV-2 complexes, if present, to produce ligation products comprising patient-specific identifying barcode sequences, and
(e) amplifying the ligation products, if present, to produce amplicons,
(f) detecting the amplicons,
wherein detection of amplicons in a pooled sample indicates that one or more of the patient samples comprises SARS-CoV-2 RNA and one or more of the patients is positive for the presence of SARS-CoV-2.

2. The method of claim 1, further comprising:
(g) performing an asymmetric RNaseH-dependent PCR on a positive pool to provide a library of nucleic acid molecules for sequencing, wherein the asymmetric PCR comprises amplification using patient-specific primers, each of which hybridizes to a patient-specific barcode sequence, and is present in approximately the same limiting concentration; and
(h) sequencing the library of nucleic acid molecules to determine the patient-specific identifier sequences, thereby identifying a SARS-CoV-2-positive patient.

3. The method of claim 1 wherein the ligating in step (d) comprises combining the pooled sample with a DNA ligase that comprises RNA-splinted DNA ligase activity.

4. The method of claim 3, wherein the DNA ligase is Chlorella virus DNA ligase PBCV-1.

5. The method of claim 1, wherein the SARS-CoV-2 RNA target sequence is in a region of the SARS-CoV-2 genome that has low secondary structure and each of the collinear oligonucleotides has a GC content from about 45% to about 55%.

6. The method of claim 1, wherein the patient-specific barcode sequence is at the 3′ end of the 5′-most collinear oligonucleotide; or is at the 5′ end 3′ of the 3′-most collinear oligonucleotide; or two of the three or more collinear oligonucleotides comprise patient-specific barcode sequences and the ligation products comprise two patient-specific barcode sequences.

7-8. (canceled)

9. The method of claim 1, wherein the amplification reaction of (d) is quantitative PCR; and/or each of the three oligonucleotides has a Tm of 55° C. or higher; and/or the oligonucleotide that hybridizes to the most 5′ position comprises the patient-specific identifier sequence and further comprises a unique molecular identifier sequence at the 5′ end of the patient-specific identifier sequence.

10-11. (canceled)

12. The method of claim 1, wherein each of the three oligonucleotides comprises one or more locked nucleic acid monomers; and/or wherein the 3′-most oligonucleotide is linked at its 5′ end to a purification moiety, optionally wherein the purification moiety is biotin.

13-14. (canceled)

15. The method of claim 1, wherein the oligonucleotide that hybridized to the most 5′ position comprises a region at the 5′ end that is not complementary to the target region of SARS-CoV-2 to which the oligonucleotide binds, but is reverse complementary to the first four nucleotides in the 3′ end that are complementary to the target region of the SARS-CoV-2 target region and form a stem-loop structure in the absence of viral template.

16. The method of claim 1, wherein the oligonucleotide that hybridizes in the 5′ position comprises the patient-specific identifier sequence and at least said 5′ most oligonucleotide is present in at least 2-fold molar excess of the SARS-CoV-2 nucleic acid.

17. The method of claim 1, further comprising a step of incubating the hybridized complex with a 5′ exonuclease after (a) and prior to (b).

18. The method of claim 1, wherein the Tm of each of the three collinear oligonucleotides is above 80° C., or wherein the Tm of each of the three collinear oligonucleotides is in the range of 60° C. to 95° C.

19. (canceled)

20. A method of rapid identification of SARS-CoV-2 positive patient(s) within a group comprising a plurality of patients in need of evaluation for SARS-CoV-2 infection comprising:
(a) separately incubating RNA-containing samples obtained from each of the plurality of patients with an oligonucleotide comprising a patient-specific barcode, wherein the oligonucleotide comprises a sequence complementary to a SARS-CoV-2 target sequence, under conditions in which, if an RNA-containing sample comprise SARS-CoV-2 genomic RNA, an oligonucleotide-SARS-CoV-2 RNA complex is produced comprising the oligonucleotide hybridized to the SARS-CoV-2 RNA,
thereby producing a plurality of incubated patient samples each of which comprises an oligonucleotide with a different patient-specific identifying barcode sequence
(b) pooling the plurality of incubated patient samples to produce a pooled sample;
(c) purifying oligonucleotide-SARS-CoV-2 RNA complexes, when present, from the pooled sample;
(d) performing a reverse transcriptase reaction to extend the oligonucleotide hybridized to the SAR-Co-V-2 RNA in the oligonucleotide-SARS-CoV-2 RNA complexes; and
(e) amplifying the ligation products, if present, to produce amplicons; and
(f) detecting the amplicons,
wherein detection of amplicons in a pooled sample indicates that one or more of the patient samples comprises SARS-CoV-2 RNA and one or more of the patients is positive for the presence of SARS-CoV-2.

21. The method of claim 20, further comprising
(g) performing an asymmetric RNaseH-dependent PCR on a positive pool to provide a library of nucleic acid molecules for sequencing, wherein the asymmetric PCR comprises amplification using patient-specific primers, each of which hybridizes to a patient-specific barcode sequence, and is present in approximately the same limiting concentration; and
(h) sequencing the library of nucleic acid molecules to determine the patient-specific identifier sequences, thereby identifying a SARS-CoV-2-positive patient.

22. The method of claim 20, wherein the SARS-CoV-2 RNA target sequence is in a region of the SARS-CoV-2 genome that has low secondary structure and the oligonucleotide has a GC content from about 45% to about 55%.

23. The method of claim 20, wherein the amplification reaction of (e) is quantitative PCR; and/or the oligonucleotide comprises one or more locked nucleic acid monomers; and/or the oligonucleotide is linked to a purification moiety, optionally biotin.

24-26. (canceled)

27. The method of claim 20, wherein the oligonucleotide comprises a region at the 5′ end that is not complementary to the target region of SARS-CoV-2 to which the oligonucleotide binds, but is reverse complementary to the first four nucleotides in the 3′ end that are complementary to the target region of the SARS-CoV-2 target region and form a stem-loop structure in the absence of viral template.

28. The method of claim 20, wherein the oligonucleotide is present in at least 2-fold molar excess of the SARS-CoV-2 nucleic acid; and/or the method further comprises a step of incubating the hybridized complex with a 3′ exonuclease prior to (d); and/or the Tm of the oligonucleotide is above 80° C. or is in the range of 65° C. to 95° C.

29-31. (canceled)

32. A method of rapid identification of single-stranded RNA (ssRNA) virus-positive patient(s) within a group comprising a plurality of patients in need of evaluation for infection with a ssRNA virus, comprising:
(a) separately incubating RNA-containing samples obtained from each of the plurality of patient with three or more collinear oligonucleotides,
wherein at least one of the three or more collinear oligonucleotides comprises a patient-specific identifying barcode sequence,
wherein the three or more collinear oligonucleotides have sequences complementary to a ssRNA virus target sequence, under conditions in which, if an RNA-containing sample comprises ssRNA virus genomic RNA, an oligonucleotide-viral RNA complex is produced comprising the three or more collinear oligonucleotides hybridized at adjacent positions in the ssRNA virus RNA target sequence,
thereby producing a plurality of incubated patient samples each of which comprises an oligonucleotide with a different patient-specific identifying barcode sequence, and
(b) pooling the plurality of incubated patient samples to produce a pooled sample;
(c) purifying oligonucleotide-viral RNA complexes, when present, from the pooled sample;
(d) ligating the three or more collinear oligonucleotides hybridized to viral RNA in the oligonucleotide-viral RNA complexes, if present, to produce ligation products comprising patient-specific identifying barcode sequences, and
(e) amplifying the ligation products, if present, to produce amplicons,
(f) detecting the amplicons,
wherein detection of amplicons in a pooled sample indicates that one or more of the patient samples comprises ssRNA virus RNA and one or more of the patients is positive for infection with the ssRNA virus.

33. The method of claim 32, further comprising:
(g) performing an asymmetric RNaseH-dependent PCR on a positive pool to provide a library of nucleic acid molecules for sequencing, wherein the asymmetric PCR comprises amplification using patient-specific primers, each of which hybridizes to a patient-specific barcode sequence, and is present in approximately the same limiting concentration; and
(h) sequencing the library of nucleic acid molecules to determine the patient-specific identifier sequences, thereby identifying a patient infected with the ssRNA virus.

34. The method of claim 32, wherein the ligating in step (d) comprises combining the pooled sample with a DNA ligase that comprises RNA-splinted DNA ligase activity, optionally wherein the DNA ligase is Chlorella virus DNA ligase PBCV-1.

35. (canceled)

36. The method of claim 32, wherein the SARS-CoV-2 RNA target sequence is in a region of the SARS-CoV-2 genome that has low secondary structure and each of the collinear oligonucleotides has a GC content from about 45% to about 55%.

37. The method of claim 32, wherein the patient-specific barcode sequence is at the 3′ end of the 5′-most collinear oligonucleotide; or the patient-specific barcode sequence at the 5′ end 3′ of the 3′-most collinear oligonucleotide; or the patient-specific barcode sequence at the 5′ end 3′ of the 3′-most collinear oligonucleotide.

38-39. (canceled)

40. The method of claim 32, wherein the amplification reaction of (e) is quantitative PCR.

41. The method of claim 32, wherein each of the three oligonucleotides has a Tm of 55° C. or higher; and/or the oligonucleotide hybridizing to the most 5′ position comprises the patient-specific identifier sequence and further comprises a unique molecular identifier sequence at the 5′ end of the patient-specific identifier sequence.

42. (canceled)

43. A method of rapid identification of ssRNA virus-positive patient(s) within a group comprising a plurality of patients in need of evaluation for infection with a ssRNA virus, comprising:
(a) separately incubating RNA-containing samples obtained from each of the plurality of patients with an oligonucleotide comprising a patient-specific barcode, wherein the oligonucleotide comprises a sequence complementary to a ssRNA virus target sequence, under conditions in which, if an RNA-containing sample comprises ssRNA virus genomic RNA, an oligonucleotide-viral RNA complex is produced comprising the oligonucleotide hybridized to the viral RNA,
thereby producing a plurality of incubated patient samples each of which comprises an oligonucleotide with a different patient-specific identifying barcode sequence;
(b) pooling the plurality of incubated patient samples to produce a pooled sample;
(c) purifying oligonucleotide-viral RNA complexes, when present, from the pooled sample;
(d) performing a reverse transcriptase reaction to extend the oligonucleotide hybridized to the viral RNA in the oligonucleotide-viral RNA complexes; and
(e) amplifying the ligation products, if present, to produce amplicons; and
(f) detecting the amplicons,
wherein detection of amplicons in a pooled sample indicates that one or more of the patient samples comprises ssRNA virus RNA and one or more of the patients is positive for the presence of ssRNA virus.

44. The method of claim 43, further comprising
(f) performing an asymmetric RNaseH-dependent PCR on a positive pool to provide a library of nucleic acid molecules for sequencing, wherein the asymmetric PCR comprises amplification using patient-specific primers, each of which hybridizes to a patient-specific barcode sequence, and is present in approximately the same limiting concentration; and
(g) sequencing the library of nucleic acid molecules to determine the patient-specific identifier sequences, thereby identifying a patient that is infected with the ssRNA virus.

45. The method of claim 43, wherein the SARS-CoV-2 RNA target sequence is in a region of the SARS-CoV-2 genome that has low secondary structure and the oligonucleotide has a GC content from about 45% to about 55%; and or the amplification reaction of (e) is quantitative PCR; and/or the Tm of the oligonucleotide is in the range of 65° C. to 95° C.

46-47. (canceled)


SEQUENCE LISTING (41 sequences)

SEQ ID NO: 1 - DNA, 18 bp, Artificial Sequence
  Description of Artificial Sequence Synthetic oligonucleotide
  Description of Combined DNA/RNA Molecule Synthetic oligonucleotide
agagcactagtcaacgaa

SEQ ID NO: 2 - DNA, 18 bp, Artificial Sequence
  Description of Artificial Sequence Synthetic oligonucleotide
  Description of Combined DNA/RNA Molecule Synthetic oligonucleotide
tgccttgatcgaacgatg

SEQ ID NO: 3 - DNA, 18 bp, Artificial Sequence
  Description of Artificial Sequence Synthetic oligonucleotide
  Description of Combined DNA/RNA Molecule Synthetic oligonucleotide
ctactcagtcagagtaga

SEQ ID NO: 4 - DNA, 18 bp, Artificial Sequence
  Description of Artificial Sequence Synthetic oligonucleotide
  Description of Combined DNA/RNA Molecule Synthetic oligonucleotide
tcgtctgactctatgtgt

SEQ ID NO: 5 - DNA, 18 bp, Artificial Sequence
  Description of Artificial Sequence Synthetic oligonucleotide
  Description of Combined DNA/RNA Molecule Synthetic oligonucleotide
gaacatacgggacaccat

SEQ ID NO: 6 - DNA, 18 bp, Artificial Sequence
  Description of Artificial Sequence Synthetic oligonucleotide
  Description of Combined DNA/RNA Molecule Synthetic oligonucleotide
cctatgactctgccaact

SEQ ID NO: 7 - DNA, 18 bp, Artificial Sequence
  Description of Artificial Sequence Synthetic oligonucleotide
  Description of Combined DNA/RNA Molecule Synthetic oligonucleotide
gagcgcaatactcgatcg

SEQ ID NO: 8 - DNA, 18 bp, Artificial Sequence
  Description of Artificial Sequence Synthetic oligonucleotide
  Description of Combined DNA/RNA Molecule Synthetic oligonucleotide
aacaaggcgtacctagcg

SEQ ID NO: 9 - DNA, 18 bp, Artificial Sequence
  Description of Artificial Sequence Synthetic oligonucleotide
  Description of Combined DNA/RNA Molecule Synthetic oligonucleotide
atgtcgtggttggatcga

SEQ ID NO: 10 - DNA, 17 bp, Artificial Sequence
  Description of Artificial Sequence Synthetic oligonucleotide
  Description of Combined DNA/RNA Molecule Synthetic oligonucleotide
ttgccgagtgtgctctc

SEQ ID NO: 11 - DNA, 24 bp, Artificial Sequence
  Description of Artificial Sequence Synthetic oligonucleotide
caagcagaagacggcatacgagat

SEQ ID NO: 12 - DNA, 25 bp, Artificial Sequence
  Description of Artificial Sequence Synthetic oligonucleotide
tggtcatctggactgctattggtgt

SEQ ID NO: 13 - DNA, 18 bp, Artificial Sequence
  Description of Artificial Sequence Synthetic primer
agagcactagtcaacgat

SEQ ID NO: 14 - DNA, 18 bp, Artificial Sequence
  Description of Artificial Sequence Synthetic primer
tgccttgatcgaacgatc

SEQ ID NO: 15 - DNA, 18 bp, Artificial Sequence
  Description of Artificial Sequence Synthetic primer
ctactcagtcagagtagt

SEQ ID NO: 16 - DNA, 18 bp, Artificial Sequence
  Description of Artificial Sequence Synthetic primer
tcgtctgactctatgtga

SEQ ID NO: 17 - DNA, 18 bp, Artificial Sequence
  Description of Artificial Sequence Synthetic primer
gaacatacgggacaccaa

SEQ ID NO: 18 - DNA, 18 bp, Artificial Sequence
  Description of Artificial Sequence Synthetic primer
cctatgactctgccaaca

SEQ ID NO: 19 - DNA, 18 bp, Artificial Sequence
  Description of Artificial Sequence Synthetic primer
gagcgcaatactcgatcc

SEQ ID NO: 20 - DNA, 18 bp, Artificial Sequence
  Description of Artificial Sequence Synthetic primer
aacaaggcgtacctagcc

SEQ ID NO: 21 - DNA, 18 bp, Artificial Sequence
  Description of Artificial Sequence Synthetic primer
atgtcgtggttggatcgt

SEQ ID NO: 22 - DNA, 18 bp, Artificial Sequence
  Description of Artificial Sequence Synthetic primer
attgccgagtgtgctctg

SEQ ID NO: 23 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagtagagcactagtcaacgat

SEQ ID NO: 24 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagttgccttgatcgaacgatc

SEQ ID NO: 25 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagtctactcagtcagagtagt

SEQ ID NO: 26 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagttcgtctgactctatgtga

SEQ ID NO: 27 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagtgaacatacgggacaccaa

SEQ ID NO: 28 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagtcctatgactctgccaaca

SEQ ID NO: 29 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagtgagcgcaatactcgatcc

SEQ ID NO: 30 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagtaacaaggcgtacctagcc

SEQ ID NO: 31 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagtatgtcgtggttggatcgt

SEQ ID NO: 32 - DNA, 58 bp, Artificial Sequence
  Description of Artificial Sequence Synthetic oligonucleotide
  Description of Combined DNA/RNA Molecule Synthetic oligonucleotide
acactctttccctacacgacgctcttccgatctgcagagtattgccgagtgtgctctg

SEQ ID NO: 33 - DNA, 60 bp, Artificial Sequence
  Description of Artificial Sequence Synthetic oligonucleotide
  modified_base (21)..(28): a, c, t, g, unknown or other
  See specification as filed for detailed description of substitutions and preferred embodiments
aatgatacggcgaccaccgannnnnnnnacactctttccctacacgacgctcttccgatc

SEQ ID NO: 34 - DNA, 44 bp, Artificial Sequence
  Description of Artificial Sequence Synthetic oligonucleotide
  modified_base (35)..(44): a, c, t, g, unknown or other
tcgagggaatttaaggtcttccttgccatgtcgannnnnnnnnn

SEQ ID NO: 35 - DNA, 54 bp, Artificial Sequence
  Description of Artificial Sequence Synthetic oligonucleotide
caagcagaagacggcatacgagattcggtagtagccaatttggtcatctggact

SEQ ID NO: 36 - DNA, 30 bp, Artificial Sequence
  Description of Artificial Sequence Synthetic oligonucleotide
gctattggtgttaattggaacgccttgtcc

SEQ ID NO: 37 - DNA, 30 bp, Severe acute respiratory syndrome coronavirus 2
tcgagggaatttaaggtcttccttgccatg

SEQ ID NO: 38 - DNA, 30 bp, Severe acute respiratory syndrome coronavirus 2; identical to SEQ ID NO: 36

SEQ ID NO: 39 - DNA, 30 bp, Severe acute respiratory syndrome coronavirus 2
tcggtagtagccaatttggtcatctggact

SEQ ID NO: 40 - DNA, 44 bp, Artificial Sequence; identical to SEQ ID NO: 34
  Description of Artificial Sequence Synthetic oligonucleotide
  modified_base (35)..(44): a, c, t, g, unknown or other

SEQ ID NO: 41 - DNA, 54 bp, Artificial Sequence; identical to SEQ ID NO: 35
  Description of Artificial Sequence Synthetic oligonucleotide